(19) United States
(12) Patent Application Publication
(10) Pub. No.: US 2026/0298897 A1
(43) Pub. Date: Oct. 1, 2026

(54) DEMULSIFIER SCREENING AND CHARACTERIZATION USING MODIFIED KARL FISCHER REAGENT

(71) Applicant: Saudi Arabian Oil Company, Dhahran (SA)

(72) Inventors: N.S. Meeran Pillai, Al-Khobar (SA); Saad M. Qahtani, Dhahran (SA); Ahmed K. AlQatari, Qatif (SA)

(21) Appl. No.: 19/096,612

(22) Filed: Mar. 31, 2025

(51) Int. Cl. G01N 31/16; C10G 33/04; G01N 33/28

(52) U.S. Cl. CPC G01N 31/168; C10G 33/04; G01N 33/2847

(57) ABSTRACT

Several demulsifiers can be screened for a crude oil and water separation characteristic, using a Karl Fischer (KF) titration technique. The KF titration technique uses a modified reagent which solubilizes a crude oil emulsion completely and rapidly releases the water molecules for titration. This improves the speed and accuracy of the KF titration technique. Further, the determination of the water content in the crude oil emulsion is used to determine the amount of wash water required in a desalter of a gas oil separation plant (GOSP).

### TECHNICAL FIELD

This disclosure relates to methods of characterizing demulsifiers.

### BACKGROUND

During oil production, both crude oil and produced water flow from the production well to the surface facilities. During flow through pumps, pipelines, fittings, chokes, and valves, the crude oil and produced water encounter sufficient mixing due to shear force. The mixing effect results in the formation of an emulsion. The emulsions can be of two types, a water-in-oil emulsion, or an oil-in-water emulsion. It is essential to destabilize the emulsion to form a crude oil phase and a water phase before the crude oil phase can be further processed and transported. Emulsions can be destabilized using demulsifiers, heat, a high electrostatic voltage, or a combination of these. Among these methods, the use of a demulsifier is the most effective method to destabilize the emulsion. However, identifying the appropriate demulsifier for each crude oil and produced water emulsion is a challenge. Demulsifier screening is a laborious manual process that typically involves visual inspection of the water and crude oil separation in a bottle. In addition, the visual inspection can be inaccurate in characterizing the demulsifier's performance, especially when the emulsion is viscous and sticks to the bottle's surface. Therefore, there is a need for an analytical method that can be used to accurately determine a demulsifier's water separation performance. Further, there is a need for an analytical method with improved efficiency that can be used to screen a large number of demulsifiers in a short time.

### SUMMARY

An implementation described herein provides a method for characterizing several demulsifiers. In some implementations, the method includes adding each of the several demulsifiers to each of the multiple oil emulsion samples; and measuring a crude oil and water separation characteristic in each of the multiple crude oil emulsion samples by a Karl Fischer (KF) titration, where the KF titration uses a modified reagent, where the modified reagent includes a mixture of organic solvents that include alcohols, cycloalkanes, aromatic solvents, or a combinations of them.

In some implementations, the modified reagent includes methanol, ethanol, propanol, butanol, long-chain alcohols, xylene, cyclohexane, cycloheptane, phenol, or a combination of them.

In some implementations, the modified reagent solubilizes each of the multiple crude oil emulsion samples.

In some implementations, the modified reagent releases water from each of the multiple crude oil emulsion samples.

In some implementations, several of the demulsifiers includes surfactant or polymer chemicals.

In some implementations, the surfactant includes ionic and non-ionic surfactants.

In some implementations, the method further includes measuring a water content in the multiple crude oil emulsion samples before adding the demulsifiers.

In some implementations, the KF titration includes a coulometric titration.

In some implementations, the method further includes after adding demulsifiers to the crude oil emulsion samples, separating the crude oil emulsion sample into a crude oil phase and a water phase.

In some implementations, the method further includes taking a sample from the crude oil phase for the KF titration to determine water content, where the sample size is about 1-20 μL.

In some implementations, the crude oil phase includes water in a range of about 0.1 ppm to 100 ppm.

An implementation described herein provides a method for determining a wash water amount in a desalter. In some implementations, the method includes producing an emulsion from an oil production well, where the emulsion includes crude oil and produced water; adding a demulsifier to the emulsion to separate the emulsion into a crude oil phase and a water phase; determining a water content in the crude oil phase using a Karl Fischer (KF) titration technique, where the KF titration technique uses a modified reagent; flowing the crude oil phase to the desalter in a gas oil separation plant (GOSP) to remove salt from the crude oil phase; determining the wash water amount needed in the desalter based on the amount of water content in the crude oil phase; and adding the wash water to the crude oil phase to remove salt.

In some implementations, the modified reagent in the KF titration technique includes alcohols, cycloalkanes, aromatic solvents, or mixtures of them.

In some implementations, the modified reagent in the KF titration technique includes methanol, ethanol, propanol, butanol, long-chain alcohols, xylene, cyclohexane, cycloheptane, phenol, or mixtures of them.

In some implementations, the KF titration technique uses a sample size from the crude oil phase in the range of about 5-20 μL.

In some implementations, the demulsifier includes an ionic or non-ionic surfactant.

An implementation described herein provides a method to determine salt content in crude oil. In some implementations, the method includes adding a demulsifier to a crude oil emulsion to separate the crude oil emulsion into crude oil and water; taking an analyte sample from the crude oil after separation of the crude oil emulsion; determining a water content in the analyte sample using a Karl Fischer titration, where the Karl Fischer titration uses a modified reagent; and determining salt content in the crude oil based on the water content in the analyte sample.

In some implementations, the modified reagent in the Karl Fischer titration includes methanol, ethanol, propanol, butanol, long-chain alcohols, xylene, cyclohexane, cycloheptane, phenol, or mixtures of them.

In some implementations, the water content in the analyte sample is in a range of about 0.1 ppm to 100 ppm.

In some implementations, the water content in the analyte sample is determined using a Karl Fischer coulometric titration.

### BRIEF DESCRIPTION OF DRAWINGS

FIG. 1 is a schematic drawing of a gas oil separation plant (GOSP).

FIG. 2 is a schematic representation of a KF titration set up.

FIG. 3 is a process flow diagram for the determination of water in a crude oil emulsion.

FIGS. 4A and 4B show the comparison of the water content determined using KF method, bottle test method, and the actual water content present in a crude oil sample.

FIG. 5 is a process flow diagram to determine the wash water required in a desalter unit in a GOSP.

### DETAILED DESCRIPTION

Implementations described herein provide a method of screening one or more demulsifiers. In some implementations, the method is used to screen 10-100 demulsifiers by accurately measuring the water separation from a crude oil emulsion, using Karl Fischer (KF) titration. In the methods of the present disclosure, a modified reagent is used in the KF titration to characterize crude oil emulsions. The modified reagent increases solubility and releases water molecules rapidly during KF titration. Implementations described herein provide a method to quantify the amount of wash water needed to desalt a crude oil using KF titration. Implementations described herein provide a method to determine the salt content in the crude oil by measuring the amount of water present in the crude oil phase using KF titration. The following description provides details for the KF titration method using the modified reagent and the determination of wash water required to desalt crude oil.

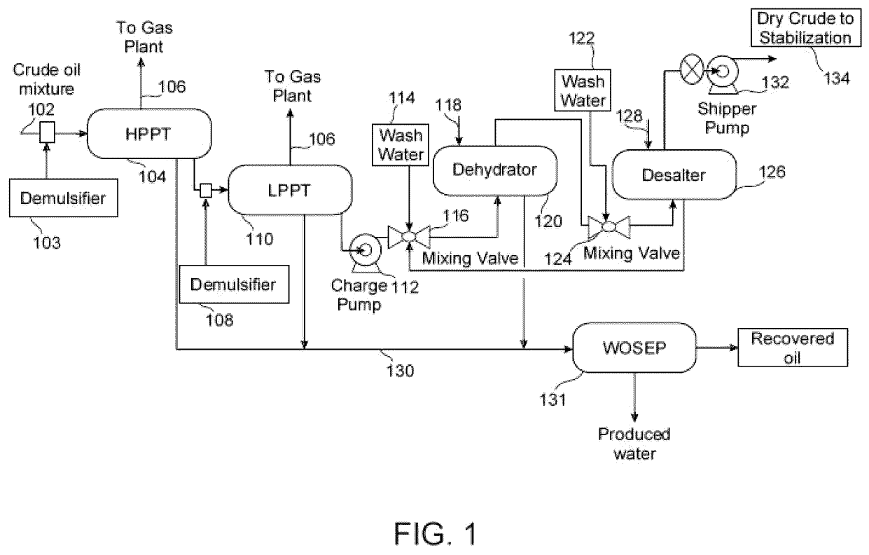

FIG. 1 is a schematic drawing of a gas oil separation plant (GOSP) that separates oil and water from an emulsion. Crude oil mixture 102, which includes oil, gas, and produced water, is produced from a production well. During flow of the crude oil mixture to a GOSP, a demulsifier 103 is dosed into the flowline. The demulsifier 103 causes the separation of water from the crude oil mixture. This helps in the downstream processing of crude oil. The crude oil mixture 102 is received by a high pressure production trap (HPPT) 104.

The HPPT 104 is a three phase separator which separates the crude oil mixture 102 into water, gas, and crude oil. In some implementations, the crude oil includes emulsified and dissolved water droplets and flows as a stable emulsion. A pressure drop in the HPPT causes the gases to be released from the crude oil. The gases released are processed in a gas plant 106. The crude oil emulsion remains in the HPPT long enough for the water to settle at the bottom. This period is known as residence time. Crude oil is separated from the crude oil emulsion. Produced water 130 is separated from the HPPT and is flowed to a water oil separator (WOSEP) 131 for further processing.

In some implementations, a second dosage of demulsifier 108 is added to the separated crude oil and is further processed by a low pressure production trap (LPPT) 110. The LPPT 110 is primarily a two phase separator. The LPPT 110 primarily removes the remaining gases from the separated crude oil by a reduction in pressure. LPPT 110 operates at about 50 psig. The separated gas from the LPPT 110 flows to a gas plant 106 for further processing. The separated crude oil from the LPPT 110 is directed to a charge pump 112. The charge pump 112 flows the separated crude oil from the LPPT through a first mixing valve 116 towards a dehydrator 120.

In some implementations, the separated crude oil is processed by the dehydrator 120 to remove the remaining water content to produce a dry crude oil. The dry crude oil contains salt and other contaminants which depends on the geological formation minerology and the formation brine originally present in the subterranean formation. These salts and contaminants are removed to prevent corrosion of the downstream processing systems. In some implementations, a first wash water stream 114 is added to the separated crude oil flowing through the first mixing valve 116. In some implementations, a third dosage of demulsifier 118 is added to the separated crude oil in the dehydrator 120 to remove any remaining water content.

The dry crude oil is flowed via a second mixing valve 124 to a desalter 126. A second wash water stream 122 is added to the dry crude oil in the desalter 126 to reduce the salinity of the dry crude oil. In some implementations, the second wash water stream 122 with a reduced salinity is obtained from fresh water sources such as ground water or desalinated sea water. The desalting process includes heating the dry crude oil along with wash water and emulsion breaking chemicals, such as a demulsifier. In some implementations, a fourth demulsifier dosage 128 is added to the dry crude oil in the desalter 126. After the desalting process, a desalted crude oil stream is obtained. In some implementations, during the desalting process a brine is obtained which contains the extracted salts and impurities. The brine obtained is corrosive and special handling materials are used to remove it from the desalter. The desalted crude oil is processed by a shipper pump 132 to a dry crude stabilization unit 134.

Demulsifiers, also known as emulsion breakers, are a class of specialty surfactant or polymer chemicals which are used to separate wet crude emulsion into a water phase and a crude oil phase. In some implementations, the demulsifiers include surfactants. The surfactants can include ionic or non-ionic surfactants. Oil field demulsifiers can include oil soluble chemicals which can break the emulsion into oil and water. In some implementations, demulsifiers include polymer intermediates of resin alkoxylates, polyols, acrylic copolymer, esters, polyglycol, poly ethylene, poly propylene oxides, and poly phenolic resins. In some implementations, demulsifiers include classes of polymers such as poly phenols, poly amines, poly alcohols, or poly oxides.

In some implementations, demulsifiers are used in crude oil dehydration and desalting processes in a gas oil separation plant (GOSP). A demulsifier's performance is characterized by a quantifiable emulsion separation index (ESI). ESI measures the amount of water separated after breaking an emulsion into oil and water. The ESI is expressed as a percentage. A high ESI value indicates a better performing demulsifier which separates the water faster. A low value of ESI indicates that the demulsifier's performance is poor and the water separation will be slower. ESI is a relative measurement of a chemical's water separation efficiency. Its value can be expressed at fixed temperatures and/or fixed demulsifier concentrations. ESI is expressed as shown in Equation 1 as:

$$ESI = \frac{\begin{matrix}\sum percentage of water separated \\ at a given time and demulsifier dosage\end{matrix}}{number of trials / readings} \qquad Eq . 1$$

Provided in the present disclosure is an analytical method using KF titration. In some embodiments, the methods of the present disclosure provide an accurate estimation of water separation from a crude oil sample. The KF titration method of the present disclosure includes the determination of water-in-crude oil before and after the addition of a demulsifier. In some implementations, an untreated crude oil emulsion (without the addition of demulsifier) has a high water content. Upon addition of the demulsifier to the crude oil emulsion, separation into a crude oil phase and water phase occurs rapidly. The KF titration measures the remaining amount of water present in the crude oil phase accurately.

In some implementations, to determine water-in-crude oil, two methods of KF titration are performed. In some implementations, the titration is volumetric titration. In some implementations, the titration is coulometric titration. In the volumetric KF titration, the titrant is directly added to the crude oil sample with the help of a burette. In some implementations, the crude oil sample is first added to a solvent. This is followed by the addition of a KF reagent which includes iodine (I₂) to the crude oil sample. Iodine can be added manually or automatically to the solvent containing the crude oil sample. The water present in the crude oil is quantified on the basis of the volume of titrant consumed. In some implementations, volumetric KF is used for the determination of water content in the range of 0.01% (100 ppm) to 100% of the sample and when there is no emulsified water

In the coulometric KF titration, the titrant is generated electrochemically in a titration cell. Coulometric titration is an absolute determination technique in which the mass of water is determined by measuring the amount of electric current required to electrolyze the water. It does not require a standard solution. In some implementations, the coulometric KF titration measures water levels much lower than the volumetric method, at values lower than 100 parts per million (ppm). In some implementations, the coulometric KF titration measures water levels as low as 10 ppm-100 ppm. In implementations herein, the coulometric titration technique traces low levels of free, emulsified, and dissolved water in the crude oil sample.

### Coulometric KF Titration Method

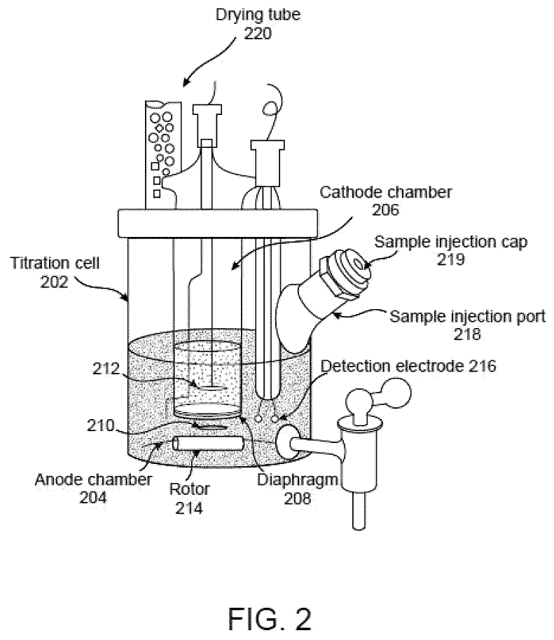

FIG. 2 is a schematic representation of a KF titration set up. A titration cell 202 includes a anode chamber 204 and a cathode chamber 206. The anode chamber 204 includes an anolyte solution. The volume of the anolyte solution can include a range of 70-100 ml solution. In some implementations, the anolyte solution includes a KF reagent. The KF reagent includes iodide ions (I⁻), sulfur dioxide (SO₂), a base, and a solvent. The base can include imidazole or pyridine. The solvents can include cycloalkanes, alcohols, or aromatic solvents. In some implementations, the alcohols include methanol, ethanol, propanol, butanol, pentanol, higher alcohols, or mixtures of them. In some implementations, the aromatic solvents include toluene, xylene, phenol, or mixtures of them. The other solvents can include cycloalkanes, such as cyclohexane or cycloheptane.

In some implementations, a catholyte solvent is used in the cathode chamber 206. Care is taken to choose a catholyte that is very dry, such that the catholyte solvent does not contribute additional water to the KF titration reaction. The catholyte is selected such that it is compatible with the anolyte. Further, the role of the catholyte is to facilitate the generation of iodine at the cathode during the titration. In some implementations, the volume of the catholyte is 25-50 mL, which can be used to run about 25 titrations. In some implementations, the catholyte solvent is used to modify the KF reagent without changing the ionic conductivity of the KF reagent.

In some implementations, the catholyte solvent includes a mixture of organic solvents. In some implementations, the KF reagent is modified by the catholyte solvent which includes a mixture of alcohols, aromatic solvents, ethers, glycols, or cycloalkanes. In some implementations, the KF reagent is modified by the catholyte solvent which includes a mixture of alcohols and aromatic solvents. In some implementations, the alcohols include methanol, ethanol, propanol, butanol, pentanol, higher alcohols, or mixtures of them. In some implementations, the aromatic solvents include xylene, phenol, toluene, or a mixture of them. In some implementations, the cycloalkanes include cyclohexane, cyclopentane, or a mixture of them.

In some implementations, the KF reagent is modified by a catholyte solvent which includes alcohol and aromatic solvents in a predetermined ratio. In some implementations, the aromatic solvents dissolve the viscous crude oil emulsion completely. In some implementations, the alcohol breaks the emulsion and rapidly releases the water from the crude oil emulsion for the reaction. Therefore, the KF reagent which is modified by the catholyte solvent improves the speed of the coulometric KF titration. Further, the KF reagent modified by the catholyte solvent (hereafter referred to as the modified KF reagent) prevents crude oil from sticking to the electrodes in the titration cell 202. Therefore, the modified KF reagent that includes an alcohol and an aromatic solvent produces a rapid and accurate measurement of the water content in the crude oil emulsion. In some implementations, the alcohol is butanol. In some implementations, the aromatic solvent is xylene. In some implementations, the alcohol is butanol and the aromatic solvent is xylene. In some implementations, the alcohol and aromatic solvent are present in a 1:1 ratio. In some implementations, butanol and xylene are present in a 1:1 ratio. In some implementations, butanol and xylene are present in other ratios. In some implementations, the modified KF reagent includes diethylene glycol or monoethyl ether.

The anode chamber 204 and the cathode chamber 206 are separated by a diaphragm 208. The diaphragm can include a ceramic material. The anode chamber has a generation electrode 210 at which the KF reactions take place. The KF titration undergoes the following reactions in the anode chamber 204.

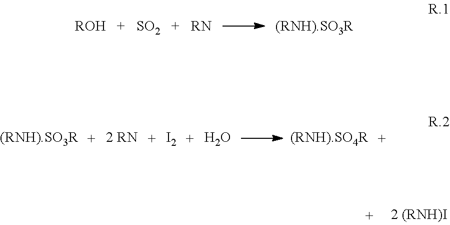

where ROH is an alcohol. In some implementations, the alcohol includes methanol, ethanol, propanol, butanol, or higher alcohols. RN is the base which can include pyridine or imidazole. In the coulometric KF titration, I₂ (titrant) is generated electrochemically from the I⁻, which occurs due to the electrolytic oxidation. When I₂ comes in contact with the water in the crude oil emulsion sample, water is titrated according to reaction R.2. Reactions R.1 and R.2 represent the chemistry behind the KF titration.

The cathode chamber 206 has an electrode 212. The electrode 212 is used to prevent the electrochemically generated I₂ from being reduced to iodide at the cathode. In some implementations, a rotor 214 is placed in the anode chamber 204 to keep the KF reactants mixed adequately. In some implementations, a detection electrode 216 is used to detect the end point of the KF reaction. The detection electrode 216 can be made of platinum. The titration cell 202 has a sample injection port 218. Through the sample injection port 218, a small volume of crude oil emulsion sample is injected into the anode chamber 204. In some implementations, the sample volume needed for the KF reaction is in the range 1-20 μL. The sample injection port 218 has a sample injection cap 219. In some implementations, the titration cell 202 has a drying tube 220. The drying tube 220 prevents ambient moisture from entering the titration cell 202.

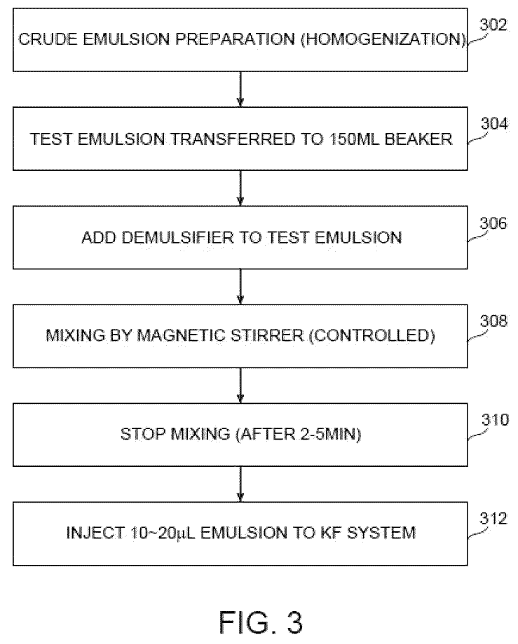

FIG. 3 is a process flow diagram for the determination of water in a crude oil emulsion. At block 302, the crude oil emulsion is prepared by homogenizing a crude oil emulsion sample. The homogenizing process helps to evenly distribute the water content in the same. In some implementations, the homogenization involves shaking the bottle for a predetermined amount of time. In some implementations, a magnetic stirrer is used to homogenize the sample.

At block 304, a volume of the homogenized crude oil emulsion sample is transferred to a 150 mL beaker. The sample volume can range between 75-100 mL. In some implementations, several samples of the homogenized crude oil emulsions are placed in different bottles. They can include samples of the same volume in each bottle.

At block 306, a demulsifier is added to the sample in one of the bottle. In some implementations, to test the performance of various demulsifiers, different demulsifiers are added to the crude oil emulsion samples in the various bottles. In some implementations, the different volumes, or weights of the same demulsifier are added to the various crude oil emulsion sample to estimate the quantity of demulsifier needed to separate water completely from the crude oil emulsion. In some implementations, various concentrations of demulsifiers are added to the multiple crude oil emulsion samples in the various bottles. This can be done to determine the optimal dosage required to separate water from the crude oil emulsion.

At block 308, once the demulsifier is added to the crude oil emulsion sample, a magnetic stirrer is used to mix the sample. This ensures adequate mixing of the demulsifiers with the crude oil emulsion samples.

At block 310, the mixing of the sample occurs for a predetermined amount of time. In some implementations, the mixing takes place for several minutes, for example in the range of about 2-10 minutes. In some implementations, the mixing takes place for hours, for example in the range of about 0.5-2 hours. In some implementations, the crude oil emulsion sample is let to rest for several minutes or hours. This causes the emulsion to separate into the crude oil phase and water phase.

At block 312, a sample from the crude oil phase (samples where demulsifier was added) is introduced via the sample injection port into the coulometric KF titration cell. In some implementations, a sample from the crude oil phase before the addition of any demulsifier is introduced into the coulometric KF titration cell to measure the initial water content. Once the initial water content is determined, performance of various demulsifiers can be measured by calculating the amount of water content remaining in the crude oil phase. The sample volume injected into the KF titration cell ranges between 1-20 μL. The coulometric KF titration of the present disclosure measures the remaining water content in the crude oil phase, by electrochemically generating iodine. The current produced during the reaction is proportional to the water content present in the crude oil phase. The current value is used to calculate the remaining water content.

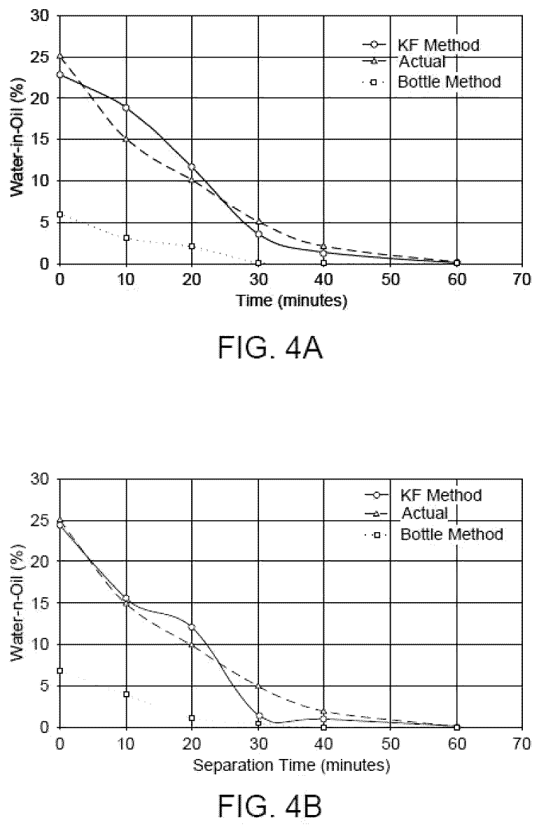

FIGS. 4A and 4B show the comparison of the water content determined using KF method, bottle test method, and the actual water content present in a crude oil sample. A crude oil emulsion sample was prepared as stated in the process flow steps of FIG. 3. The crude oil emulsion samples were obtained from a production well. The crude oil emulsion samples were homogenized by shaking it using an automated shaker. After homogenization of the crude oil emulsion sample, a test sample of a predetermined volume 75-100 mL was transferred to a bottle.

A particular demulsifier was chosen for the KF titration. The demulsifier was added at a dosage of 10 ppm-100 ppm of the total sample volume to the test crude oil emulsion sample. The test crude oil emulsion sample and the demulsifier were mixed using a magnetic stirrer for a period of 2-5 minutes. The addition of a demulsifier to the test crude oil emulsion sample initiated water separation from the crude oil emulsion sample. This resulted in a crude oil phase and a water phase due to the separation effect. After waiting for a predetermined amount of time, for example 5-30 min, about 10-20 μL of sample (hereafter referred to as sample A) was siphoned out of the separated crude oil phase.

Sample A was introduced into the sample injection port of the KF titration cell. Sample A was introduced into the anode chamber which included the modified KF reagent. The modified KF reagent included I⁻ ions, SO₂, imidazole as the base, and butanol and xylene in the ratio 1:1 as the solvent. I₂ was generated electrochemically in the anode chamber of the KF titration cell. The KF titration follows the reaction scheme as shown in R.1 and R.2. The electric current produced to generate the I₂ was used to calculate the amount of water content remaining in the crude oil phase of the test crude oil emulsion sample.

FIG. 4A represents Run #1 of the comparison of the different methods to determine water content in the crude oil emulsion sample after the addition of a demulsifier. The curve represented by the legend actual was the data from a sample which had a predetermined amount of water added to the oil sample. The water separation effect or the percentage of water present in the oil sample was studied as a function of time and concentration of demulsifier.

In some implementations, the actual sample is also known as standard sample, which can be used for calibration of the KF titration cell. However, in most cases a coulometric sample does not need a standard sample for calibration. The curve represented by the legend KF method in FIG. 4A was the data measured using the coulometric KF titration method in the present disclosure. The KF titration method used the modified KF reagent. It is to be noted that the data represented by the KF method is very close to the data represented by the actual water content present in the crude oil sample. The curve represented by the legend bottle method was the data measured by making visual observations of water separation as a function of time for the oil emulsion sample, after the addition of a demulsifier. The results of the bottle test departed significantly from the actual results.

FIG. 4B represents Run #2 of the comparison of the results for the same oil sample used in Run #1. Run #2 was conducted to check for reproducibility. Run #2 data also shows that the curve represented by the KF method was very close to the curve represented by the actual data in comparison with the curve represented by the bottle method. The data of the bottle method deviated significantly from the data represented by the actual sample. Therefore, both FIGS. 4A and 4B demonstrate that the KF method of the present disclosure is accurate in determining the water content present in the crude oil emulsion.

In some crude oil emulsion samples, water exists in the crude oil as dissolved water or emulsified water. The coulometric KF method of the present disclosure is suitable, especially in such cases. Further, coulometric KF method of the present disclosure can detect water at very low ranges, such as less than 100 ppm. In some implementations, the coulometric KF titration of the present disclosure detects water in the range of 0.1 to 10 ppm.

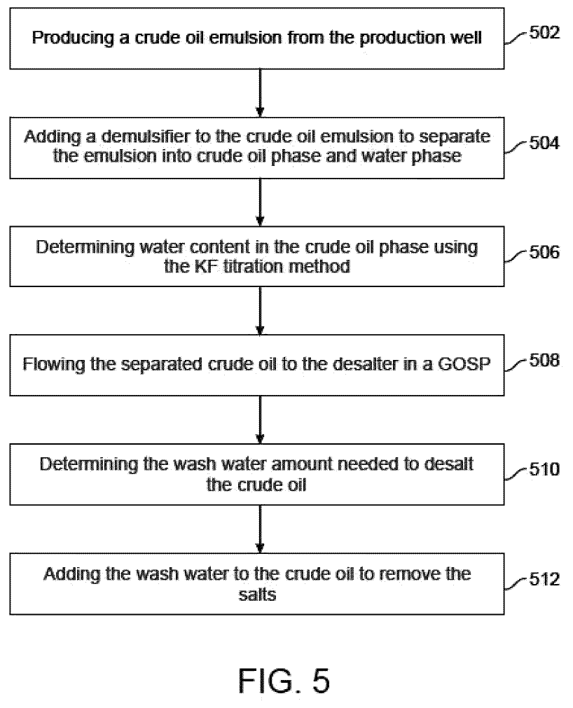

FIG. 5 is a process flow diagram to determine the wash water required in a desalter unit in a GOSP. At block 502, an emulsion is produced from the production well. In oil and gas production, crude oil is produced along with produced water. As there is multi-phase flow during oil production, the fluids that flow through the pipelines, valves, and various fittings experience turbulence and mixing. This mixing gives rise to the formation of emulsions of water-in-crude oil or crude oil-in-water. Emulsions are generally viscous fluids.

At block 504, a demulsifier is added to the flowing emulsion. In some implementations, several dosages of demulsifiers are added at multiple locations during crude oil emulsion flow. Multiple dosages at various points help to separate more water from the crude oil before the crude oil enters the desalter unit in a GOSP. In some implementations, different demulsifier chemistries and concentrations are added at various points depending on the water content present in flowing crude oil emulsion. The presence of water retains more salt in the crude oil. The presence of salt causes corrosion to the downstream pipelines and processing facilities. Further, for the safety of crude oil transportation, the presence of corrosive material like salt and metals is not preferred.

At block 506, upon adding the demulsifier to the flowing crude oil emulsion, separation of the emulsion into a crude oil phase (hereafter referred to as crude oil) and water phase occurs. In some implementations, the emulsion is heated to further evaporate the water from the crude oil emulsion. In some implementations, high voltage electrostatic field is applied to remove the water from the emulsion. In some implementations, a combination of a chemical demulsifier, heat, and electrostatic voltage is used.

At various points during flow of the crude oil, demulsifiers are dosed into the flowing crude oil pipeline. The crude oil flows into the dehydrator to remove any remaining water present. In some implementations, there is water still left in the crude oil after dehydration. The effluent stream from the dehydrator is a dry crude oil stream. In some implementations, samples of the dry crude oil stream are prepared for KF titration analysis. KF titration method is used to determine the remaining water content in the dry crude oil, before flowing it to the desalter unit. KF titration method can measure water content as low as 10-100 ppm. In some implementations, a coulometric KF titration method is used to determine the water content in the dry crude oil.

In some implementations, the KF reagent is modified to adapt to the crude oil measurement. In some implementations, the KF reagent includes iodide ions, SO₂, a base, and a solvent. In some implementations, the base includes imidazole or pyridine. In some implementations, the solvents include alcohols, cycloalkanes, aromatic solvents, or a combinations of them. In some implementations, the solvents include methanol, ethanol, propanol, butanol, long-chain alcohols, xylene, cyclohexane, cycloheptane, phenol, or mixtures of them. In some implementations, the solvent includes butanol and xylene in the ratio 1:1 to solubilize the crude oil completely. Further, butanol rapidly releases the water molecules from the crude oil, which gives fast and accurate measurements in the KF titration method.

At block 508, the dry crude oil is flowed into a desalter unit of a GOSP. The presence of water retains salt in the dry crude oil. Therefore, the measured water content using the KF titration method provides an estimation of the amount of salt retained in the crude oil.

At block 510, wash water is used to dilute the salt content in the crude oil. In some implementations, wash water is obtained from fresh sources such as ground water. In some implementations, the source of wash water is the recycled permeate stream from a desalination unit. In some implementations, a combination of ground water and a recycled permeate stream is used as a source of wash water. In some implementations, these sources of wash water include 5000-10,000 ppm of total dissolved solids (TDS). Therefore, a fresh water source is used as a wash water stream to dilute the salt content and remove them from the crude oil. The amount of wash water needed to desalt the crude is determined from the amount of salt present in the crude oil.

At block 512, the wash water is added to the crude oil to remove the salt. A discharge brine containing various impurities from the crude oil such as inorganic salt and metals are removed from the desalter unit. The discharge brine can be dumped into a well or further purified to be used as an injection water.

The implementations described herein provide a method to determine the water content in a crude oil accurately and effectively using the KF titration method, where the KF titration reagent is modified to produce fast and accurate results. Viscous crude oil emulsions are not soluble in commercial KF reagents. Therefore, the water in the crude oil emulsion is not determined accurately using commercial KF reagents. Further, the commercial KF reagents give inaccurate results as the viscous crude oil sticks to the electrode surface. The water content is used to determine the desalting efficiency of a desalter in a GOSP. The implementations described here provide a method to determine the amount of salt present in the crude oil by determining the amount of water present in the crude oil. Implementations described herein provide a method to measure the amount of wash water required to desalt a crude oil.

Other implementations are also within the scope of the following claims.

### EXEMPLARY EMBODIMENTS

1. A method of characterizing a plurality of demulsifiers comprising:
adding each of the plurality of demulsifiers to each of a plurality of crude oil emulsion samples; and
measuring a crude oil and water separation characteristic in each of the plurality of crude oil emulsion samples by a Karl Fischer (KF) titration, wherein the KF titration uses a modified reagent, wherein the modified reagent comprises a mixture of organic solvents comprising alcohols, cycloalkanes, aromatic solvents, or combinations thereof.

2. The method of embodiment 1, wherein the modified reagent comprises methanol, ethanol, propanol, butanol, long-chain alcohols, xylene, cyclohexane, cycloheptane, phenol, or combinations thereof.

3. The method of embodiment 1 or 2, wherein the modified reagent solubilizes each of the plurality of crude oil emulsion samples.

4. The method of any of embodiments 1-3, wherein the modified reagent releases water from each of the plurality of crude oil emulsion samples.

5. The method of any of embodiments 1-4, wherein the plurality of demulsifiers comprises surfactant or polymer chemicals.

6. The method of any of embodiments 1-5, wherein the surfactant comprises ionic and non-ionic surfactants.

7. The method of any of embodiments 1-6, further comprising measuring a water content in the plurality of crude oil emulsion samples before adding the plurality of demulsifiers.

8. The method of any of embodiments 1-7, wherein the KF titration comprises a coulometric titration.

9. The method of any of embodiments 1-8, further comprising, after adding a plurality of demulsifiers to the respective plurality of crude oil emulsion samples, separating the plurality of crude oil emulsion samples into a crude oil phase and a water phase.

10. The method of any of embodiments 1-9, further comprising taking a sample from the crude oil phase for the KF titration to determine water content, wherein the sample size comprises about 1-20 μL.

11. The method of any of embodiments 1-10, wherein the crude oil phase comprises water in a range of about 0.1 ppm to 100 ppm.

12. A method of determining a wash water amount in a desalter, the method comprising:
producing an emulsion from an oil production well, wherein the emulsion comprises crude oil and produced water;
adding a demulsifier to the emulsion to separate the emulsion into a crude oil phase and a water phase;
determining a water content in the crude oil phase using a Karl Fischer (KF) titration technique, wherein the KF titration technique uses a modified reagent;
flowing the crude oil phase to the desalter in a gas oil separation plant (GOSP) to remove salt from the crude oil phase;
determining the wash water amount needed in the desalter based on the amount of water content in the crude oil phase; and
adding the wash water to the crude oil phase to remove salt.

13. The method of embodiment 12, wherein the modified reagent in the KF titration technique comprises alcohols, cycloalkanes, aromatic solvents, or mixtures thereof.

14. The method of embodiment 12 or 13, wherein the modified reagent in the KF titration technique comprises methanol, ethanol, propanol, butanol, long-chain alcohols, xylene, cyclohexane, cycloheptane, phenol, or mixtures thereof.

15. The method of any of embodiments 12-14, wherein the KF titration technique uses a sample size from the crude oil phase in the range of about 5-20 μL.

16. The method of any of embodiments 12-15, wherein the demulsifier comprises an ionic or non-ionic surfactant.

17. A method to determine salt content in crude oil, the method comprising:
adding a demulsifier to a crude oil emulsion to separate the crude oil emulsion into crude oil and water;
taking an analyte sample from the crude oil after separation of the crude oil emulsion;
determining a water content in the analyte sample using a Karl Fischer titration, wherein the Karl Fischer titration uses a modified reagent; and
determining salt content in the crude oil based on the water content in the analyte sample.

18. The method of embodiment 17, wherein the modified reagent in the Karl Fischer titration comprises methanol, ethanol, propanol, butanol, long-chain alcohols, xylene, cyclohexane, cycloheptane, phenol, or mixtures thereof.

19. The method of embodiment 17 or 18, wherein the water content in the analyte sample is in a range of about 0.1 ppm to 100 ppm.

20. The method of any of embodiments 17-19, wherein the water content in the analyte sample is determined using a Karl Fischer coulometric titration.

1. A method of characterizing a plurality of demulsifiers comprising:
adding each of the plurality of demulsifiers to each of a plurality of crude oil emulsion samples; and
measuring a crude oil and water separation characteristic in each of the plurality of crude oil emulsion samples by a Karl Fischer (KF) titration, wherein the KF titration uses a modified reagent, wherein the modified reagent comprises a mixture of organic solvents comprising alcohols, cycloalkanes, aromatic solvents, or combinations thereof.

2. The method of claim 1, wherein the modified reagent comprises methanol, ethanol, propanol, butanol, long-chain alcohols, xylene, cyclohexane, cycloheptane, phenol, or combinations thereof.

3. The method of claim 2, wherein the modified reagent solubilizes each of the plurality of crude oil emulsion samples.

4. The method of claim 2, wherein the modified reagent releases water from each of the plurality of crude oil emulsion samples.

5. The method of claim 1, wherein the plurality of demulsifiers comprises surfactant or polymer chemicals.

6. The method of claim 5, wherein the surfactant comprises ionic and non-ionic surfactants.

7. The method of claim 1, further comprising measuring a water content in the plurality of crude oil emulsion samples before adding the plurality of demulsifiers.

8. The method of claim 1, wherein the KF titration comprises a coulometric titration.

9. The method of claim 1, further comprising, after adding a plurality of demulsifiers to the respective plurality of crude oil emulsion samples, separating the plurality of crude oil emulsion samples into a crude oil phase and a water phase.

10. The method of claim 9, further comprising taking a sample from the crude oil phase for the KF titration to determine water content, wherein the sample size comprises about 1-20 μL.

11. The method of claim 10, wherein the crude oil phase comprises water in a range of about 0.1 ppm to 100 ppm.

12. A method of determining a wash water amount in a desalter, the method comprising:
producing an emulsion from an oil production well, wherein the emulsion comprises crude oil and produced water;
adding a demulsifier to the emulsion to separate the emulsion into a crude oil phase and a water phase;
determining a water content in the crude oil phase using a Karl Fischer (KF) titration technique, wherein the KF titration technique uses a modified reagent;
flowing the crude oil phase to the desalter in a gas oil separation plant (GOSP) to remove salt from the crude oil phase;
determining the wash water amount needed in the desalter based on the amount of water content in the crude oil phase; and
adding the wash water to the crude oil phase to remove salt.

13. The method of claim 12, wherein the modified reagent in the KF titration technique comprises alcohols, cycloalkanes, aromatic solvents, or mixtures thereof.

14. The method of claim 13, wherein the modified reagent in the KF titration technique comprises methanol, ethanol, propanol, butanol, long-chain alcohols, xylene, cyclohexane, cycloheptane, phenol, or mixtures thereof.

15. The method of claim 12, wherein the KF titration technique uses a sample size from the crude oil phase in the range of about 5-20 μL.

16. The method of claim 12, wherein the demulsifier comprises an ionic or non-ionic surfactant.

17. A method to determine salt content in crude oil, the method comprising:
adding a demulsifier to a crude oil emulsion to separate the crude oil emulsion into crude oil and water;
taking an analyte sample from the crude oil after separation of the crude oil emulsion;
determining a water content in the analyte sample using a Karl Fischer titration, wherein the Karl Fischer titration uses a modified reagent; and
determining salt content in the crude oil based on the water content in the analyte sample.

18. The method of claim 17, wherein the modified reagent in the Karl Fischer titration comprises methanol, ethanol, propanol, butanol, long-chain alcohols, xylene, cyclohexane, cycloheptane, phenol, or mixtures thereof.

19. The method of claim 17, wherein the water content in the analyte sample is in a range of about 0.1 ppm to 100 ppm.

20. The method of claim 19, wherein the water content in the analyte sample is determined using a Karl Fischer coulometric titration.
